(19) United States
(12) Patent Application Publication
(10) Pub. No.: US 2004/0180427 A1
(43) Pub. Date: Sep. 16, 2004

(54) Dually operating medical apparatus for safe sampling and inoculation

(76) Inventor: Mao-Kuei Chang, Taipei (TW)

Correspondence Address: Mao-Kuei Chang, P. O. Box 55-846, Taipei, 104, TW

(21) Appl. No.: 10/794,481

(22) Filed: Mar. 2, 2004

(30) Foreign Application Priority Data
Mar. 14, 2003 (TW) 92203937

(51) Int. Cl. C12M 1/26

(52) U.S. Cl. 435/309.1; 435/288.1; 435/304.1

(57) ABSTRACT

A medical apparatus includes a sampling and inoculating device detachably mounted in a container; having a suction portion formed on the sampling and inoculating device for sucking a liquid specimen which is then discharged into the container for storage or transport, a swabbing head detachably secured to a lower portion of the sampling and inoculating device for picking up the specimen and also for primarily inoculating the specimen onto a culture dish for a streak procedure, and an inoculating head formed on the lower portion of the sampling and inoculating device for subsequently diluting and separating the bacteria from the zone as streaked by the swabbing head for well inoculating and culturing the bacteria in order to identify the bacteria for a reliable medical treatment.

### BACKGROUND OF THE INVENTION

U.S. Pat. No. 5,091,316 to Monthony et al. disclosed a microbiological culture collection and transport device including a swabbing tip (11) formed on one end of a shaft (10) having a swab cover (13) covering the swabbing tip (11) and a sample inoculator (15) having an inoculator cover (14) covering the inoculator (15). However, such a prior art has the following drawbacks:

1. When the swabbing tip (11) is inserted into the tubular cover (13), it must be done very carefully. For inserting the tip (11) into such a tiny tubular cover (13), trace of specimen may easily contaminate the cover (13) and the cap (12). After inverting the swabbing tip (11) upwardly and turning the inoculator (15) downwardly for inoculation, the laboratorian when holding the tubular cover (14, 13) may be easily contaminated or even be infected by the bacteria or virus existing in the specimen.

2. The swabbing tip (11), even clad with sterile polyurethane foam, may only collect little amount of specimen. It lacks of any suction mechanism so that it may not suck much liquid specimen such as urine, sputum and snivel. Excess saturation of a liquid sample by the polyurethane foam on the swabbing tip (11), the specimen liquid may be dripped to cause pollution or contamination to the surrounding.

3. The tubular cover 13 may only store little amount of specimen, possibly influencing the biological examination.

The present inventor has found the drawbacks of the prior art and invented this dually operating medical apparatus for safe sampling and inoculation.

### SUMMARY OF THE INVENTION

The object of the present invention is to provide a medical apparatus including a sampling and inoculating device detachably mounted in a container; having a suction portion formed on the sampling and inoculating device for sucking a liquid specimen which is then discharged into the container for storage or transport, a swabbing head detachably secured to a lower portion of the sampling and inoculating device for picking up the specimen and also for primarily inoculating the specimen onto a culture dish for a streak procedure, and an inoculating head formed on the lower portion of the sampling and inoculating device for subsequently diluting and separating the bacteria from the zone as streaked by the swabbing head for well inoculating and culturing the bacteria in order to identify the bacteria for a reliable medical treatment.

### BRIEF DESCRIPTION OF THE DRAWINGS

FIG. 1 is a perspective view of the present invention.

FIG. 2 is an exploded view of the present invention.

FIG. 3 is a partially enlarged view showing the lower portion of the sampling and inoculating device from FIG. 2.

FIG. 4 is a perspective view of the sampling and inoculating device of the present invention.

FIG. 5 is a sectional drawing of the present invention when assembled.

FIG. 6 shows a primary inoculation by the swabbing head of the present invention.

FIG. 7 shows the removal of the swabbing head in accordance with the present invention.

FIG. 8 shows a secondary inoculation by the inoculating head (following FIG. 6) of the present invention.

FIG. 9 shows a tertiary inoculation of the present invention following FIG. 8.

FIG. 10 shows a quaternary inoculation of the present invention following FIG. 9.

FIG. 11 is a bottom view of the inoculating head of the present invention.

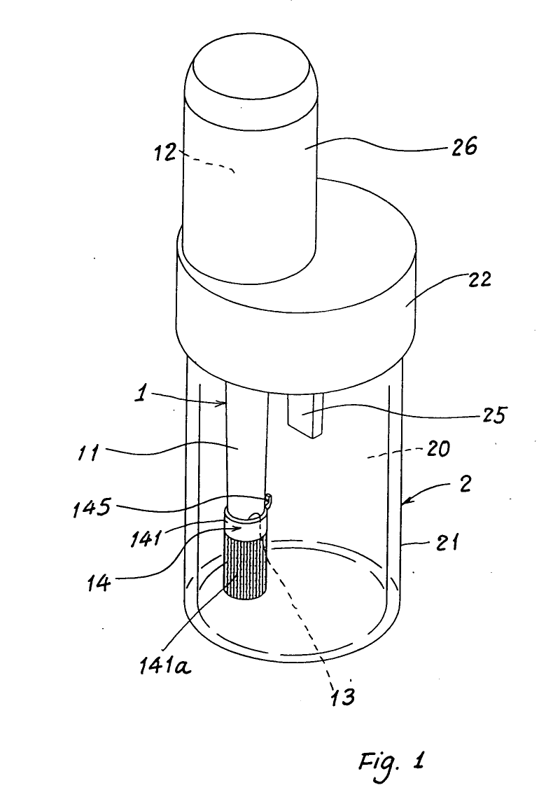

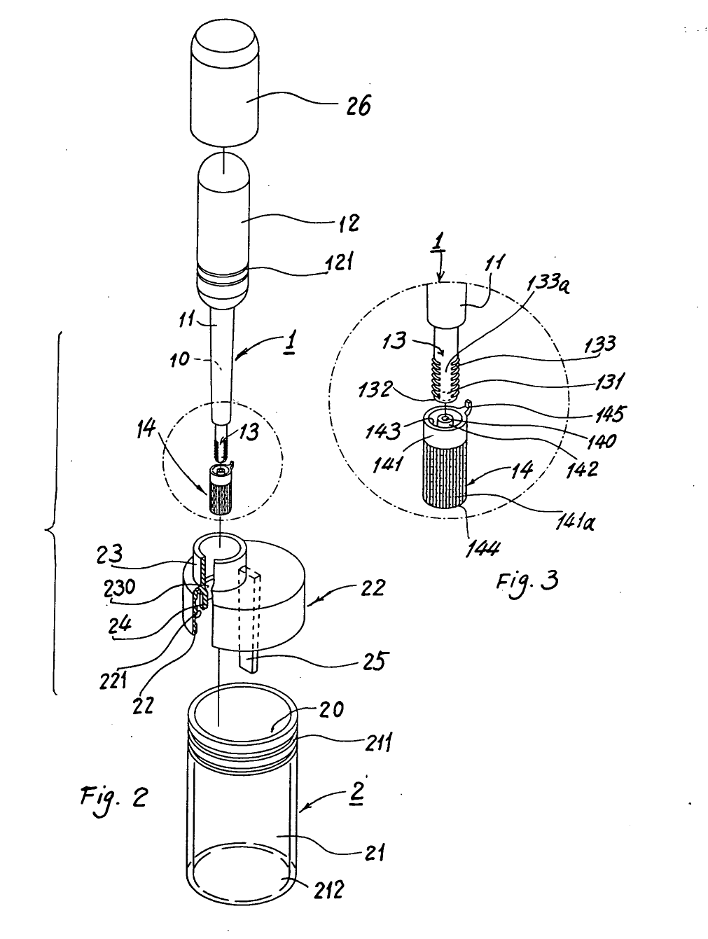

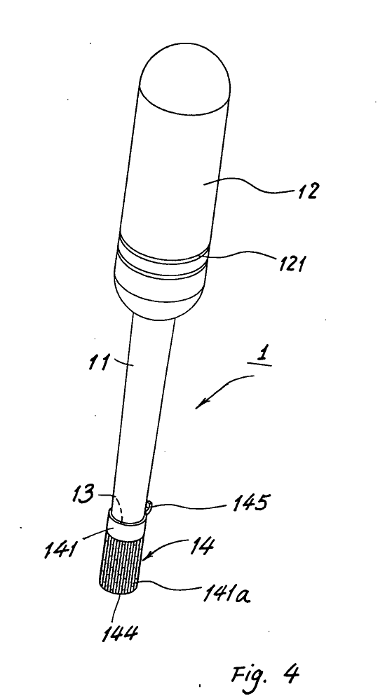

### DETAILED DESCRIPTION

As shown in the drawing figures, the dually operating medical apparatus of the present invention comprises: a sampling and inoculating device 1, and a container 2.

The sampling and inoculating device 1 includes: a hollow stem 11, a suction (or pumping) portion 12 formed on a top of the hollow stem 11, an inoculating head 13 formed on a lower portion of the hollow stem 11 and a swabbing head 14 detachably secured to a bottom portion of the hollow stem 11 and concentrically juxtapositionally disposed around the inoculating head 13.

The sampling and inoculating device 1 is detachably mounted in the container 2. As shown in the figures, the sampling and inoculating device 1 is eccentrically mounted on the container 2 for a convenient pick-up of specimen as stored in the container 2, especially when tilted.

The suction portion 12 may be formed as a flexible suction bulb made of rubber or plastic materials, having a diameter larger than the diameter of the hollow stem 11.

The suction portion 12 includes a plurality of flexible extension rings 121 annularly formed on a cylindrical surface of the suction portion 12 to be engaged with a plurality of circular grooves 241 annularly recessed in a coupling sleeve 24 of the container 2 for detachably mounting the sampling and inoculating device 1 on the container 2.

The hollow stem 11 is tapered lengthwise downwardly and is integrally connected with the inoculating head 13 which may be generally cylindrically shaped.

The inoculating head 13 includes a central opening 131 formed through the inoculating head 13 to be fluidically communicated with a tubular interior 10 defined within the hollow stem 11, and a bottom rim portion 132 formed on a bottom of the inoculating head 13.

The inoculating head 13 further includes a plurality of discontinuous projections 133 circumferentially formed on a cylindrical surface 133a of the inoculating head 13, having the discontinuous projections 133 of the inoculating head 13 frictionally engaging with the swabbing head 14 when detachably securing the swabbing head 14 to the bottom portion of the hollow stem 11 (FIG. 5), with the discontinuous projections 133 on the cylindrical surface 133a allowing air or gas passing therethrough.

The swabbing head 14 includes: an outer cylindrical portion 141 frictionally engaged with the projections 133 of the inoculating head 13, an axial tubular pin 142 axially protruding upwardly from a circular bottom disk 144 formed on a bottom of the swabbing head 14 and insertably engaged with the central opening 131 formed in the inoculating head 13, an annular chamber 143 defined between the outer cylindrical portion 141 and the axial tubular pin 142, a central hole 140 formed through the axial tubular pin 142 and through the bottom disk 144 and fluidically communicated with the tubular interior 10 in the hollow stem 11; whereby upon insertion of the axial tubular pin 142 into the central opening 131 in the inoculating head 13, the swabbing head 14 will be detachably secured to the inoculating head 13 and also detachably secured to the hollow stem 11, defining a gas-flow passage in between the cylindrical surface 133a of the inoculating head 13 and the outer cylindrical portion 141 of the swabbing head 14. The projections 133 of the inoculating head 13 help frictionally hold the outer cylindrical portion 141 of the swabbing head 14 to thereby detachably secure the swabbing head 14 to the inoculating head 13 stably.

The swabbing head 14 includes an absorbent layer 141a such as made of cotton formed, clad or coated on the outer cylindrical portion 141 for absorbing specimen onto the swabbing head 14 for collecting, smearing or disposing the specimen.

The swabbing head 14 further includes a dismantling extension (or lug) 145 protruding sidewardly from the swabbing head 14 to be thrusted and removed by a pusher rod 25 formed in the container 2 (FIG. 7).

When mounting the sampling and inoculating device 1 on the container 2, the inoculating head 13 and the swabbing head 14 are approximating the bottom 212 of a bottle portion 21 of the container 2.

The swabbing head 14 may be integrally formed as an absorbent member such as made of cotton or porous liquid-absorbable material which is detachably secured to the inoculating head 13 on the hollow stem 11.

The container 2 includes: a bottle portion 21 preferably made as transparent cylindrical bottle, a cover 22 secured on an upper portion of the bottle portion 21, a hopper portion 23 formed on the cover 22 having a hopper interior 230 confined within the hopper portion 23 for smoothly receiving the sampling and inoculating device 1 therein for detachably mounting the sampling and inoculating device 1 on the container 2.

The hopper portion 23 is protruded upwardly from the cover 22 to be disposed around the suction portion 12 of the sampling and inoculating device when detachably mounting the sampling and inoculating device 1 on the container 2 (FIG. 5), having a cap 26 engaging with the hopper portion 23 for covering the suction portion 12 of the sampling and inoculating device 1 within the container 2. The hopper portion 23 is formed with at least an extension ring 231 thereon to be engaged with a ring groove 261 circularly recessed in the cap 26.

The container 2 includes a pusher rod 25 protruding downwardly from the cover 22 into an interior 20 within the bottle portion 21 for thrusting and removing the swabbing head 14 into said container as shown in FIG. 7 for inoculation as effected by the inoculating head 13.

The hopper portion 23 further includes a coupling sleeve 24 tapered downwardly from the hopper portion 23 for detachably holding the suction portion 12 of the sampling and inoculating device 1 when mounted on the container 2, having at least one groove 241 circularly recessed in the coupling sleeve 24 to be engaged with at least an extension ring 121 formed on the suction portion 12 of the sampling and inoculating device 1. The bottle portion 21 has its upper portion formed with male threads 211 for engaging female threads 221 formed in the cover 22 for securing the cover 22 on the container 2.

When using the sterile apparatus of the present invention, the sampling and inoculating device 1 is detached from the container 2 for collecting a patient's specimen including sputum, snivel, urine, etc. The suction portion 12 may be operated to suck the liquid specimen into the hollow stem 11. The specimen is then discharged into the container 2 for storage or transport. The swabbing portion 14 containing the absorbent layer 141a will adhere or absorb the specimen thereon.

The sampling and inoculating device 1 may be sterilized in advance, such as being sterilized by ethylene oxide (EO) gas, to kill all bacteria before sampling and inoculation. The EO gas may enter the chamber 143 in the head 14 and into interior 10 in the stem 11 for a thorough sterilization of the device 1 before inoculation.

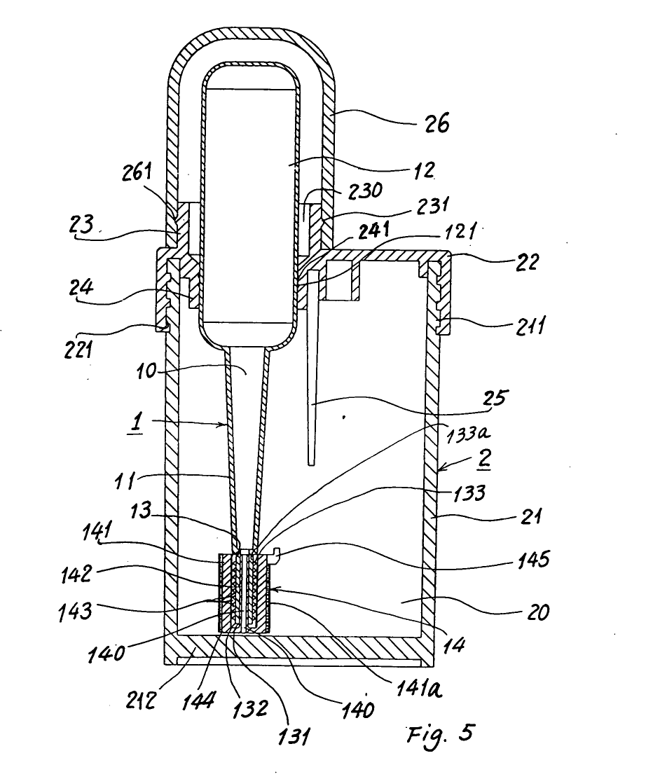

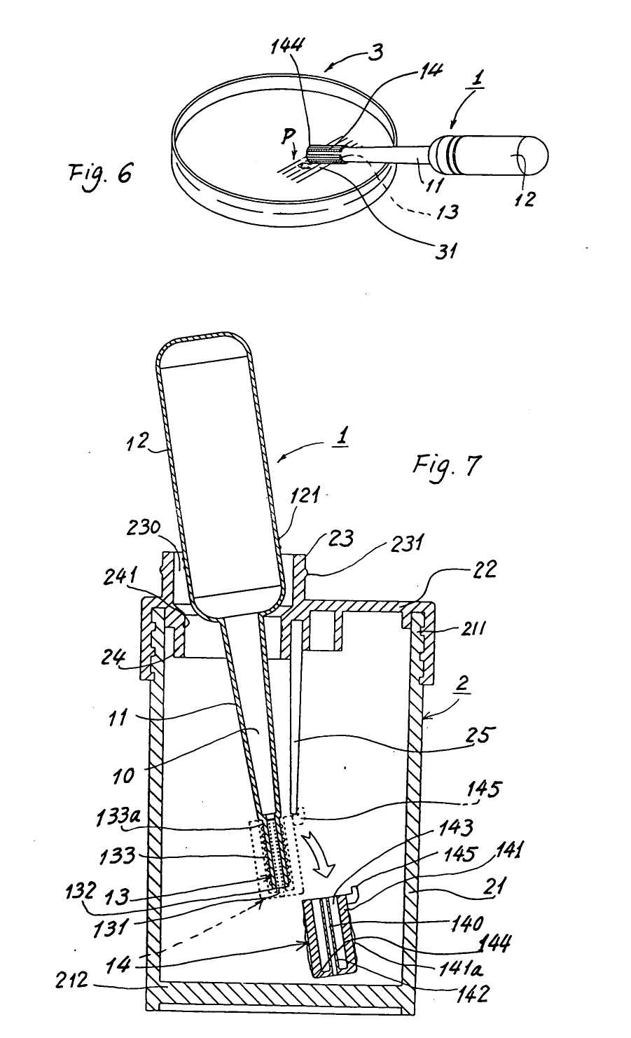

When it is intended to culture bacteria from the specimen as stored in the container 2, the sampling and inoculating device 1 is removed outwardly from the container 2 to allow the bottom disk 144 of the swabbing head 14 to streak for plural parallel lines 31 on a culture dish 3 as shown in FIG. 6 to conduct a primary inoculation (P) by smearing specimen on the dish 3.

Then the swabbing head 14 is removed and detached into the container 2 by the pusher rod 25 as shown in FIG. 7 to expose the inoculating head 13.

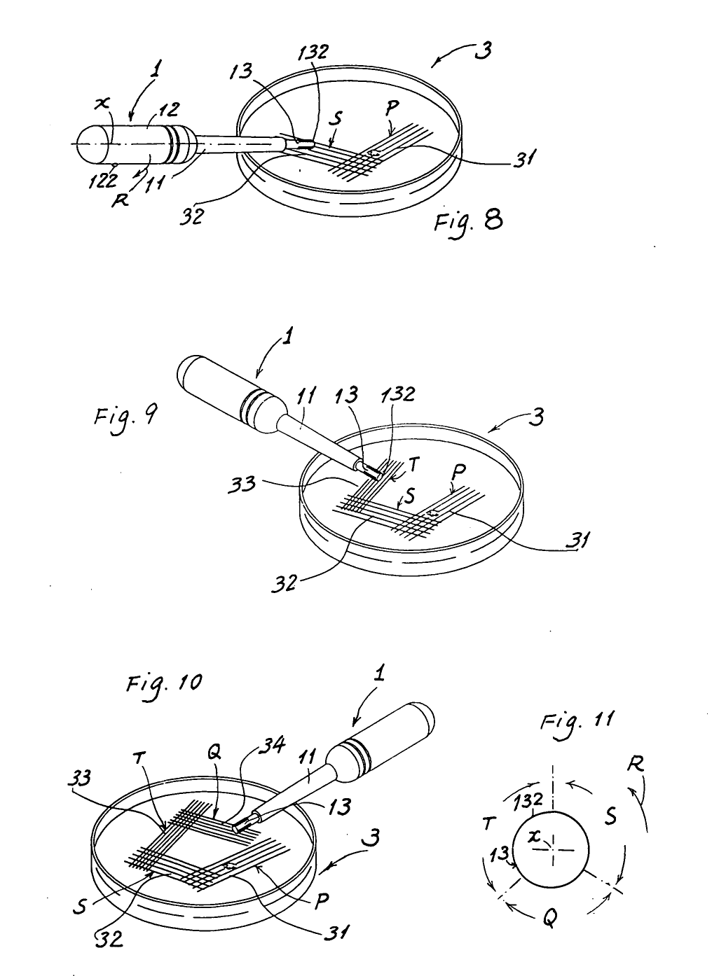

The sampling and inoculating device 1 is then held to allow the bottom rim portion 132 of the inoculating head 13 to streak several parallel lines 32 on the culture dish 3 as shown in FIG. 8 to conduct a secondary inoculation (S). During such a streaking procedure, the bacteria will be diluted and separated from the primary inoculation zone (P) to the secondary inoculation zone (S).

The device 1 is held and rotated (R) about the axis X of the stem 11 to change the peripheral portion of the bottom rim portion 132 of the inoculating head 13 (FIG. 11) from the secondary inoculation zone (S) to the tertiary zone (t) to continuously streak further parallel lines 33 as shown in FIG. 9 to conduct the tertiary inoculation (T). By the way, the bacteria will be further diluted and separated from the previous step as shown in FIG. 8.

Further rotation (R) of the device 1 to change the bottom rim portion 132 of the inoculating head 13 and upon a further streaking of the inoculating head 13 to draw lines 34 on the dish 3 from the tertiary inoculation (T) to the quaternary inoculation (Q) as shown in FIG. 10.

Accordingly, four-zone streak procedures will be completed to dilute and separate the bacteria, which is cultured to form colony for medical examination in order to identify the bacterial specy for a reliable medical treatment.

During the streak procedure such as from FIG. 6 through FIG. 10, the sterile inoculating head 13 and the device 1 of the present invention is no longer sterilized to prevent any contamination hazard by spreading the virus or bacteria from the specimen on the instrument to the laboratorian. Otherwise, a conventional inoculator should be repeatedly sterilized by high-temperature sterilization. The “cold” specimen on the conventional inoculator when heated may be sputtered to the laboratorian to cause dangerous infection or pollution. The present invention has omitted such a high-temperature sterilization and will keep the related people in a safer working environment.

As shown in FIG. 11, the bottom rim portion 132 of the inoculating head 13 is “divided” into three peripheral portions respectively corresponding to a secondary inoculation (S) as shown in FIG. 8, a tertiary inoculation (T) as shown in FIG. 9, and a quaternary inoculation (Q) in FIG. 10. The primary inoculation (P) has been previously done by the swabbing head 14 as shown in FIG. 6. A marking 122 as shown in FIG. 8 may be formed on the device 1 for sensing the positioning of the inoculating head 13 during the streak procedures by rotating the device 1.

Therefore, the present invention is superior to the prior art with the following advantages:

1. Repeated sterilizations are eliminated to prevent contamination of the patient's specimen to the related workers and laboratorians.

2. Just holding the upper portion of the device, the sampling of specimen and the inoculation may be safely done without inverting the device.

3. A suction mechanism as effected by the suction portion 12 on the hollow stem 11 is provided to efficiently collect a liquid sample.

4. The container 2 provides an enough storage device for storing a large amount of specimen, being beneficial for an ergonomic sampling and inoculation jobs.

5. A simple device as disclosed in this invention may be used for four-zone streak for diluting and separating the bacteria in a simpler safer way for facilitating the culturing of bacteria.

6. The sampling and inoculation can be accomplished by a single device to reduce the cost for implementing laboratory equipments.

The present invention may be modified without departing from the spirit and scope of the present invention.

I claim:

1. A medical apparatus comprising:
a container; and
a sampling and inoculating device detachably mounted in and on said container, said sampling and inoculating device including: a hollow stem, a suction portion formed on a top of the hollow stem for sucking liquid specimen into said hollow stem, an inoculating head formed on a lower portion of the hollow stem for inoculation, and a swabbing head, for collecting specimen thereon and for swabbing the specimen to a culture dish, detachably secured to a bottom portion of the hollow stem and concentrically juxtapositionally disposed around the inoculating head.

2. An apparatus according to claim 1, wherein said suction portion on said sampling and inoculating device is formed as a flexible suction bulb, having a diameter of said suction portion larger than a diameter of the hollow stem.

3. An apparatus according to claim 2, wherein said suction portion includes a plurality of flexible extension rings annularly formed on a cylindrical surface of the suction portion to be engaged with a plurality of circular grooves annularly recessed in a coupling sleeve formed in a container for detachably mounting the sampling and inoculating device on the container.

4. An apparatus according to claim 1, wherein said inoculating head is formed as cylindrical shape and including a central opening formed through the inoculating head to be fluidically communicated with a tubular interior defined within the hollow stem, and a bottom rim portion formed on a bottom of the inoculating head.

5. An apparatus according to claim 4, wherein said inoculating head further includes a plurality of discontinuous projections circumferentially formed on a cylindrical surface of the inoculating head, having the discontinuous projections of the inoculating head frictionally engaging with a swabbing head when detachably securing the swabbing head to a bottom portion of the hollow stem, with the discontinuous projections on the cylindrical surface allowing air or gas passing therethrough.

6. An apparatus according to claim 1, wherein said swabbing head includes: an outer cylindrical portion frictionally engaged with a plurality of projections formed on a cylindrical surface of the inoculating head, an axial tubular pin axially protruding upwardly from a circular bottom disk formed on a bottom of the swabbing head and insertably engaged with a central opening formed in the inoculating head, an annular chamber defined between the outer cylindrical portion and the axial tubular pin, a central hole formed through the axial tubular pin and through the bottom disk and fluidically communicated with a tubular interior in the hollow stem insertable in said container; whereby upon insertion of the axial tubular pin into the central opening in the inoculating head, the swabbing head will be detachably secured to the inoculating head and detachably secured to the hollow stem, defining a gas-flow passage in between the cylindrical surface of the inoculating head and the outer cylindrical portion of the swabbing head.

7. An apparatus according to claim 6, wherein said swabbing head includes an absorbent layer formed on the outer cylindrical portion for absorbing specimen onto the swabbing head for collecting or disposing the specimen.

8. An apparatus according to claim 6, wherein said swabbing head further includes a dismantling extension protruding sidewardly from the swabbing head to be thrusted and removed by a pusher rod formed in the container.

9. An apparatus according to claim 1, wherein said swabbing head is integrally formed as an absorbent member made of porous liquid-absorbable material which is detachably secured to the inoculating head on the hollow stem.

10. An apparatus according to claim 1, wherein said container includes: a bottle portion, a cover secured on an upper portion of the bottle portion, and a hopper portion formed on the cover having a hopper interior confined within the hopper portion for receiving the sampling and inoculating device therein for detachably mounting the sampling and inoculating device on the container.

11. An apparatus according to claim 10, wherein said hopper portion is protruded upwardly from the cover to be disposed around a suction portion of the sampling and inoculating device when detachably mounting the sampling and inoculating device on the container, having a cap engaging with the hopper portion for covering the suction portion of the sampling and inoculating device within the container.

12. An apparatus according to claim 10, wherein said container includes a pusher rod protruding downwardly from the cover into an interior within the bottle portion for thrusting and removing the swabbing head into said container for inoculation as effected by an inoculating head.

13. An apparatus according to claim 10, wherein said hopper portion further includes a coupling sleeve tapered downwardly from the hopper portion for detachably holding a suction portion of the sampling and inoculating device when mounted on the container, having at least a groove circularly recessed in the coupling sleeve to be engaged with at least an extension ring formed on the suction portion of the sampling and inoculating device.

14. An apparatus according to claim 1, wherein said inoculating head includes a bottom rim portion divided into a plurality of peripheral portions, each said peripheral portion corresponding to a streak procedure when conducting inoculation by said inoculating head on a culture dish.

15. An apparatus according to claim 6, wherein said swabbing head when detachably mounted on said inoculating head defines an annular air passage between said inoculating head and said swabbing head to enter a sterilizing gas into said annular air passage for sterilization therein.
